(19) United States
(12) Patent Application Publication
(10) Pub. No.: US 2009/0130233 A1
(43) Pub. Date: May 21, 2009

(54) Two part lotion

(76) Inventors: Christopher G. Baker, Port Jervis, NY (US); Robert D. Kross, Bellmore, NY (US)

Correspondence Address: KLAUBER & JACKSON, 411 HACKENSACK AVENUE, HACKENSACK, NJ, 07601, US

(21) Appl. No.: 12/290,012

(22) Filed: Oct. 24, 2008

Related U.S. Application Data
(60) Provisional application No. 61/000,237, filed on Oct. 24, 2007.

(51) Int. Cl. A61K 33/00; A61P 31/04; A61P 31/12; A61P 31/10; A61P 33/00

(52) U.S. Cl. 424/718

(57) ABSTRACT

The present invention provides a composition, substantially a lotion, containing a source of nitrite ions and water. The composition, substantially a lotion, is preferably provided in two distinct and separate formulations. One of these formulations may or may not contain a source of nitrite ions. The composition is easily washed off with soap and water and is useful in preventing or treating bacterial, viral, parasitic or fungal infections. Further, the invention provides methods for preventing or treating bacterial, viral, parasitic or fungal infections by applying a pharmaceutically effective amount of the composition topically to the skin. Still further, the invention provides a kit comprising the composition of according to the invention together with instructions for applying the composition topically.

### FIELD OF THE INVENTION

The present invention relates generally to a composition, substantially a skin lotion containing a source of nitrite ions useful as an anti-bacterial, anti-parasitic, anti-viral or anti-fungal agent. The present invention further provides methods, compositions and kits useful for treating or preventing bacterial, viral or fungal infections on the skin surface or for reducing the number of microorganisms on a skin surface.

### BACKGROUND

A growing body of evidence suggests expanded use of nitric oxide based systems in the control of microorganisms, especially those microbes pathogenic to human beings. Nitrates, nitrites, and inorganic nitrogen containing acids have all been employed to control microbes, especially bacteria. Agricultural and food related applications of this technology have emerged and been commercialized over the last several decades.

The use of such systems has also influenced the medical community, and in recent years many experiments have been performed to demonstrate the effectiveness of nitric oxide produced in vivo (via acidified nitrite-containing urine present in the human bladder) in treating Urinary Tract Infections (UTI). Nitric oxide based systems for controlling microorganisms, thereby enhancing human health and well being, is thus a well established practice.

There is a void in the area of direct human topical treatments (i.e., dermatologic compounds) in the otherwise highly active industrial, scientific, and medical research being conducted on nitric oxide. The reasons for the lack of activity in dermatological and topical applications center around the inherent instability of such systems when delivered in forms suitable for direct application to the human skin. The highly volatile and highly reactive nature of nitric oxides make it impossible to deliver in a simple cream, ointment, or lotion that would commonly provide the vehicle for a pharmaceutical active.

However, delivery of nitric oxide to the skin, particularly the teats, of certain farm animals has been considered, researched, and even implemented commercially several years ago.

It would therefore be useful to provide a topical delivery system capable of delivering a pharmaceutically active quantity of nitric oxide or a precursor thereof effective as an anti-microbial on the skin. Consideration of such systems led to an avenue of research designed to produce a personal use product that would deliver sufficient nitric oxide, and its inevitable by-products, to cause a minimum of a three log reduction in microbial content on human skin.

U.S. Patent Publication 20050142218 describes pharmaceutical compositions containing nitrite source and an acidifying agent for treating skin ischaemia. The use of acidified nitrate as an agent to produce local production of nitrate oxide at the skin surface is described in the treatment of peripheral ischaemia and associated conditions.

U.S. Patent Publication 20050037093 describes treatment of nail infections with NO. The applicants contend that nitrogen oxide generating compositions are useful in the treatment of subungual infections, as NO has surprisingly been found to be able to penetrate the nail to exert an anti-fungal effect.

U.S. Patent Publication 20050036949 describes a pharmaceutical dispenser, comprising a liquid formulation comprising nitric oxide, and means for forming a nebulised mist of the liquid formulation. The nebulised mist of the liquid formulation is used primarily in the treatment of respiratory diseases.

U.S. Patent Publication 20040105898 describes a dosage form for the treatment of bacterial, viral or fungal conditions which comprises, a pharmaceutically acceptable acidifying agent in an amount sufficient to reduce the pH at an environment of use to below pH4, and a pharmaceutically acceptable source of nitrite ions or a nitrate precursor therefor; where the acidifying agent and source of nitrite ions or nitrate precursor are separately disposed in respective pharmaceutically acceptable carriers for admixture at the intended environment of use to release NO or NO₂ ions. The publication also describes delivery systems for the topical medicament.

U.S. Patent Publication 20040037897 describes that the products of the acidification of nitrite are useful to control multiply drug resistant bacteria, such as methicillin resistant S. aureus.

U.S. Patent Publication 20040013747 describes a pharmaceutical delivery system comprising a pharmaceutically active agent and acidified nitrite as an agent to produce local production of nitric oxide at the skin surface. The dosage form may be in any pharmaceutically acceptable carrier means and comprises an acidifying agent adapted to reduce the pH at the environment. In one embodiment, a barrier consisting of a membrane allows diffusions of the anesthetic and nitrite ions while preventing direct contact of the skin and acidifying agent.

U.S. Patent Publication 20020155174 and 20020136750 describe acidified nitrite as an antimicrobial agent and provides a dosage form for the treatment of bacterial, viral or fungal conditions which comprises a pharmaceutically acceptable acidifying agent in an amount sufficient to reduce the pH at an environment of use to below pH4, and a pharmaceutically acceptable source of nitrite ions or a nitrate precursor. The acidifying agent and the source of nitrite ions or nitrate precursor are separately disposed in respective pharmaceutically acceptable carriers for admixture at the intended environment of use to release NO or NO₂ ions. The invention also provides delivery systems for the topical medicament.

U.S. Patent Publication 20020090401 describes pharmaceutical composition containing nitrate source and an acidifying agent for treating skin ischaemia The use of acidified nitrate as an agent to produce local production of nitrate oxide at the skin surface is described in the treatment of peripheral ischaemia and associated conditions. The dosage form may be in any pharmaceutically acceptable carrier means and comprises an acidifying agent adapted to reduce the pH at the environment. A barrier consisting of a membrane allows diffusions of the nitrate ions while preventing direct contact of the skin and acidifying agent. The applicants contend that the invention is useful for managing chronic skin wounds and peripheral ischaemia conditions such as Raynaud's phenomenon.

Likewise, U.S. Pat. No. 6,709,681 describes a dosage form for the treatment of bacterial, viral or fungal conditions which comprises a pharmaceutically acceptable acidifying agent in an amount sufficient to reduce the pH at an environment of use to below pH4, and a pharmaceutically acceptable source of nitrite ions or a nitrate precursor therefore. The acidifying agent and the source of nitrite ions or nitrate precursor are separately disposed in respective pharmaceutically acceptable carriers for admixture at the intended environment of use to release NO or NO₂ ions. The invention also provides delivery systems for the topical medicament.

### SUMMARY OF THE INVENTION

In a first aspect, the present invention provides a composition that is substantially a lotion. The composition preferably has a neutral olfactory sensation. The viscosity of the composition is in the range of between 5,000 centipoise and 200,000 centipoise. Additionally, the composition is soluble in soap and water. The composition is useful in the prevention and treatment of bacterial, viral, parasitic or fungal infections. Further, the composition is effective, easy to apply and to remove.

The composition typically contains at least one agent that functions in four, preferably five, six, seven, eight or more of the following categories as a Skin protectant, Solvent/diluent, Humectant/moisturizer, Skin soother, Emollient, polymer/colloid stabilizer, Slip/texture agent, Preservative, Surfactant, Buffering agent, optionally an Antiseptic, optionally an Acidifying agent, optionally a source of nitrite ions, and optionally a colorant. In especially preferred embodiments, the composition features at least one agent that functions in each of the following categories as a Skin protectant, Solvent/diluent, Humectant/moisturizer, Emollient, polymer/colloid stabilizer, Acidifying agent, Slip/texture agent, Preservative, Surfactant, Buffering agent. In these preferred embodiments, there may optionally be an agent that functions as an Antiseptic, optionally be an agent that functions as a Skin soother, optionally an agent that functions as a source of nitrite ions, and optionally a colorant.

The skin protectant agents include such as allantoin and dimethicone which function to, among other things, moisturize, increase keratolysis, increase desquamation, provide a barrier to environmental stressors, and form complexes with irritants.

The antiseptic agents include benzalkonium chloride which function as antimicrobial agents.

The Solvent and diluent is normally water, especially deionized water. It is normally present in an amount of QS to 100%.

The humectant or moisturizer may be glycerin, butylene glycol and the like.

The skin soother may be benzyl alcohol which functions to, among other things, reduce erythema and produce an anesthetic effect.

The emollient may be caprylic/capric triglyceride.

The polymer/colloid stabilizer may be acrylamide/sodium acryloyldimethyltaurate copolymer, polyacrylamide and the like.

The acidifying agent may be lactic acid. The acidifying agent is adapted to reduce the pH at the site of application and can include any suitable organic acid such as ascorbic acid (vitamin C), salicylic acid, acetyl salicylic acid, acetic acid or a salt or a derivative thereof in a concentration up to 20% w/w, suitably 0.25 to 10% w/w, preferably 0.50 to 3% w/w. A particularly preferred concentration is 1% or 2% w/w. The preferred pH range is from pH2 to pH7, preferably pH2 to pH3. Other acidifying agents include but are not limited to, ammonium or aluminium salts, phenol, benzoic acid. Inorganic acids such as hydrochloric acid may be used if sufficient dilute and/or appropriately buffered. The acidifying agent may be present as a dissolved salt or in a liquid form.

The Slip/texture agent may be isohexadecane or C13-14 isoparaffin and the like which function to

The preservative may be methylparaben, phenoxyethanol, butylparaben, propylparaben, isobutylparaben and polyquaternium-15 and the like. These compounds function to inhibit the growth of bacteria and to prevent the degradation of the product that would occur in their absence.

The surfactant may be, for instance, polysorbate 80, laureth-7, polyglyceryl-4 isostearate, Cetyl PEG/PPG 10/1 dimethicone and the like.

The buffering agent may be, for instance, citric acid, ascorbic acid, sodium hydroxide, and the like.

Deionized water acts as a vehicle/solvent for the other ingredients. The deionized water is present in the amount which maximizes ease of application of the product, by giving it a consistency that allows the lotion to be easily spread onto the skin. Further, the deionized water serves to maintain the proper hydration to the skin.

The pharmacologically acceptable source of nitrite ions may be an alkaline metal nitrite or an alkaline earth metal nitrite, For example, LiNO₂, NaNO₂, KNO₂, RbNO₂, CsNO₂, FrNO₂, Be(NO)₂, Mg(NO₂)₂, Ca(NO₂)₂, Sr(NO₂)₂, Ba(NO₂)₂, or Ra(NO₂)₂. Alternatively, a nitrite precursor may be used as the source of the nitrite ions in the composition, such as for example a dilute solution of nitrous acid. Other sources of nitrite ions are nitrate ions derived from alkali metal or alkaline earth metal salts capable of enzymic conversion to nitrite. For example, LiNO₃, NaNO₃, KNO₃, RbNO₃, CsNO₃, FrNO₃, Be(NO₃)₂, Mg(NO₃)₂, Ca(NO₃)₂, Sr(NO₃)₂, Ba(NO₃)₂ or Ra(NO₃)₂. The concentration of the nitrite ion source may be up to 20% w/w, suitably 0.10 to 10%, preferably 0.25 to 1%. A particularly preferred concentration is 0.40% or 0.50% w/w. The pharmacologically acceptable sources of nitrite ions or a nitrite precursor, e.g., nitrates, will be generally referred to herein as the “pharmacologically acceptable source of nitrite” or “pharmacologically acceptable source of nitrite ions.”

In preferred embodiments of the invention the skin protectant is present in a concentration of about 0.1 to 5% by weight, preferably 0.25 to 3%, or 0.50 to 2.50% by weight. In other preferred embodiments of the invention the Humectant/moisturizer is present in a concentration of about 0.1 to 20% by weight, preferably 1 to 15%, or 2 to 10%, or 5 to 8% by weight. In preferred embodiments of the invention the Emollient is present in a concentration of about 0.1 to 20% by weight, preferably 0.5 to 10%, or 1 to 2% by weight. In additional preferred embodiments of the invention the polymer/colloid stabilizer is present in a concentration of about 0.1 to 20% by weight, preferably 0.5 to 10%, or 1 to 7%, or 2.5 to 5.0, especially preferably about 3.0 to 4.0% by weight. In still further preferred embodiments of the invention the Slip/texture agent is present in a concentration of about 0.1 to 20% by weight, preferably 0.25 to 10%, or 0.50 to 2.50% by weight. In yet other preferred embodiments of the invention the Preservative is present in a concentration of about 0.1 to 20% by weight, preferably 0.25 to 10%, or 0.50 to 2.50%, or 0.75 to 1.00% by weight. In yet additional preferred embodiments of the invention the Surfactant is present in a concentration of about 0.05 to 15% by weight, preferably 0.25 to 10%, or 0.30 to 2.50%, or 0.40 to 0.75% by weight. In more preferred embodiments of the invention the Buffering agent is present in a concentration of about 0.01 to 5% by weight, preferably 0.25 to 10%, or 0.30 to 2.50%, or 0.40 to 0.75% by weight. In still other preferred embodiments of the invention the optional Antiseptic is present in a concentration of about 0.01 to 5% by weight, preferably 0.05 to 2%, or 0.10 to 1.00%, or 0.25 to 0.75% by weight. In additional preferred embodiments of the invention the optional skin soother is present in a concentration of about 0.01 to 5% by weight, preferably 0.05 to 10%, or 0.10 to 5.00%, or 0.50 to 2.00% by weight. In even further preferred embodiments of the invention the optional acidifying agent is present in a concentration of about 0.1 to 15% by weight, preferably 0.25 to 10%, or 0.50 to 5.00%, or 1.00 to 2.00% by weight. In yet other preferred embodiments of the invention the optional source of nitrite ions is present in a concentration of about 0.01 to 10% by weight, preferably 0.10 to 5%, or 0.25 to 2.00%, or 0.40 to 1.00% by weight. In some preferred embodiments of the invention the optional colorant is present in a concentration of about 0.01 to 2% by weight, preferably 0.01 to 1%, or 0.01 to 0.10%, or 0.01 to 0.05% by weight.

The composition, substantially a lotion may optionally contain additional ingredients such as aloe vera, well documented moisturizing and moisture balancing properties as well as bactericidal properties and kills bacteria before they can enter skin. Additionally, Vitamin C, chemically known as ascorbic acid, is important in the synthesis of collagen, an important structural component of skin. Consequently, vitamin C is vital for skin regeneration and is also a powerful antioxidant. Additional antioxidizing agents may include, for instance, soybean oil and alpha lipoic acid. Soybean oil is actually a mixture of substances, and it acts as an antioxidant by preventing free radicals from forming and damaging cells and tissues. Alpha lipoic acid is a natural molecule found in every living cell of the human body. It is a powerful antioxidant and anti-inflammatory compound.

In preferred embodiments, more than one, preferably two distinct and separate formulations are provided together. One formulation may or may not contain the source of nitrite ions. According to these embodiments, a first composition comprises a source of nitrite ions. In preferred embodiments of the invention the skin protectant is present in a concentration of about 0.1 to 5% by weight, preferably 0.25 to 3%, or 0.50 to 2.50% by weight. In other preferred embodiments of the invention the humectant/moisturizer is present in a concentration of about 0.1 to 20% by weight, preferably 1 to 15%, or 2 to 10%, or 5 to 8% by weight. In preferred embodiments of the invention the Emollient is present in a concentration of about 0.1 to 20% by weight, preferably 0.5 to 10%, or 1 to 2% by weight. In additional preferred embodiments of the invention the polymer/colloid stabilizer is present in a concentration of about 0.1 to 20% by weight, preferably 0.5 to 10%, or 1 to 7%, or 2.5 to 5.0, especially preferably about 3.0 to 4.0% by weight. In still further preferred embodiments of the invention the Slip/texture agent is present in a concentration of about 0.1 to 20% by weight, preferably 0.25 to 10%, or 0.50 to 2.50% by weight. In yet other preferred embodiments of the invention the Preservative is present in a concentration of about 0.1 to 20% by weight, preferably 0.25 to 10%, or 0.50 to 2.50%, or 0.75 to 1.00% by weight. In yet additional preferred embodiments of the invention the Surfactant is present in a concentration of about 0.05 to 15% by weight, preferably 0.25 to 10%, or 0.30 to 2.50%, or 0.40 to 0.75% by weight. In more preferred embodiments of the invention the Buffering agent is present in a concentration of about 0.01 to 5% by weight, preferably 0.25 to 10%, or 0.30 to 2.50%, or 0.40 to 0.75% by weight. In yet other preferred embodiments of the invention the source of nitrite ions is present in a concentration of about 0.01 to 10% by weight, preferably 0.10 to 5%, or 0.25 to 2.00%, or 0.40 to 1.00% by weight. In some preferred embodiments of the invention the colorant is present in a concentration of about 0.01 to 2% by weight, preferably 0.01 to 1%, or 0.01 to 0.10%, or 0.01 to 0.05% by weight. In preferred embodiments, the pH of this composition is between about 6.0 and 10.0, preferably between about 7.0 and 8.0, and in some preferred embodiments between about 7.2 and 7.8, for example between 7.3 and 7.6.

According to these embodiments of the invention, a second composition does not comprise a substantial source of nitrite ions. In preferred embodiments of the invention the skin protectant is present in a concentration of about 0.1 to 5% by weight, preferably 0.25 to 3%, or 0.50 to 2.50% by weight. In other preferred embodiments of the invention the humectant/moisturizer is present in a concentration of about 0.1 to 20% by weight, preferably 1 to 15%, or 2 to 10%, or 5 to 8% by weight. In preferred embodiments of the invention the emollient is present in a concentration of about 0.1 to 20% by weight, preferably 0.5 to 10%, or 1 to 2% by weight. In additional preferred embodiments of the invention the polymer/colloid stabilizer is present in a concentration of about 0.1 to 20% by weight, preferably 0.5 to 10%, or 1 to 7%, or 2.5 to 5.0, especially preferably about 3.0 to 4.0% by weight. In still further preferred embodiments of the invention the Slip/texture agent is present in a concentration of about 0.1 to 20% by weight, preferably 0.25 to 10%, or 0.50 to 2.50% by weight. In yet other preferred embodiments of the invention the Preservative is present in a concentration of about 0.1 to 20% by weight, preferably 0.25 to 10%, or 0.50 to 2.50%, or 0.75 to 1.00% by weight. In yet additional preferred embodiments of the invention the surfactant is present in a concentration of about 0.05 to 15% by weight, preferably 0.25 to 10%, or 0.30 to 2.50%, or 0.40 to 0.75% by weight. In more preferred embodiments of the invention the buffering agent is present in a concentration of about 0.01 to 5% by weight, preferably 0.25 to 10%, or 0.30 to 2.50%, or 0.40 to 0.75% by weight. In still other preferred embodiments of the invention the optional Antiseptic is present in a concentration of about 0.01 to 5% by weight, preferably 0.05 to 2%, or 0.10 to 1.00%, or 0.25 to 0.75% by weight. In additional preferred embodiments of the invention the skin soother is present in a concentration of about 0.01 to 5% by weight, preferably 0.05 to 10%, or 0.10 to 5.00%, or 0.50 to 2.00% by weight. In even further preferred embodiments of the invention the acidifying agent is present in a concentration of about 0.1 to 15% by weight, preferably 0.25 to 10%, or 0.50 to 5.00%, or 1.00 to 2.00% by weight. In yet other preferred embodiments of the invention the source of nitrite ions is present in a concentration of about 0.01 to 10% by weight, preferably 0.10 to 5%, or 0.25 to 2.00%, or 0.40 to 1.00% by weight. In some preferred embodiments of the invention the colorant is present in a concentration of about 0.01 to 2% by weight, preferably 0.01 to 1%, or 0.01 to 0.10%, or 0.01 to 0.05% by weight. In preferred embodiments, the pH of this composition is between about 1.0 and 5,0, preferably between about 2.0 and 4.0, and in some preferred embodiments between about 2.2 and 2.4. In especially preferred embodiments, the first composition comprising a source of nitrite ions comprises buffered lactic acid as an acidifying agent, and the second composition comprising no substantial source of nitrite ions comprises NaNO. The compositions of the present invention are preferably biologically stable so as to maintain substantially all or least about 90% of their ability to kill an organism for at least a week, preferably at least a month, more preferably at least a year, and even more preferably at least two years under room temperature and pressure conditions.

In a second aspect, the invention provides a method for treating or preventing one or more of bacterial, viral, parasitic or fungal infections by applying a pharmaceutically effective amount of a composition, substantially a lotion according to the invention to the skin surface of a mammal, especially a human. In other aspects, the invention provides a method for reducing the number of bacterial, viral, parasitic or fungal organisms on the surface of the skin by applying a pharmaceutically effective amount of a composition, substantially a lotion according to the invention to the skin surface of a mammal, especially a human. In preferred embodiments, the number of organisms on a surface, such as, for instance, the skin are reduced on the order of at least 2 log₁₀, 3 log₁₀, 4 log₁₀, 5 log₁₀ or even 6 log₁₀ or 7 log₁₀. That is, the number of organisms on a surface, such as, for instance, the skin are reduced on the order of at least 50%, 60%, 70%, 80%, 90%, 95%, 98%, 99%, 99.5%, 99.9%, 99.99% or even 99.999%. In some instances, the number of organisms present on a surface, such as, for instance, the skin are reduced to a substantially undetectable level.

In some embodiments the subject organism is a bacteria, in some embodiments the subject organism is a virus, in some embodiments the subject organism is a yeast, and in some embodiments the subject organism is a fungus. The methods of the present invention may be useful against one, two, three, four, five or even ten or twenty or fifty or more identifiable microorganisms simultaneously. In some embodiments, the methods of the present invention are effective against at least half, at least two-thirds, at least three-quarters or even substantially all identifiable microorganisms simultaneously. Some representative organisms that the methods of the present invention may be useful in killing include, for instance, Escherichia coli, Staphylococcus aureus, Pseudomonas aeruginosa, and Candida albicans.

The methods of the present invention may be practiced by applying the compositions of the present invention directly to a surface. The surface may include the skin or mucosa, a medical device or implant or surgical instrument, or any surface desired to be sanitized or even sterilized. The compositions may be applied for any suitable length of time such as, for instance, at least 10 seconds, at least 30 seconds, at least 45 seconds, at least 1, 2, 3, 5, 7, 10, 15 or even 20 or 30 minutes, or the compositions may be applied to a surface and not removed at all until such time as they naturally wear off, evaporate, or are washed off. The compositions may be applied with a hand or the fingers, gloved or without gloves, or with a special applicator, or from a tube or bottle, etc. adapted to dispense the compositions.

In a third aspect, the invention provides a kit comprising a composition according to the invention housed in one or more containers together with instructions for using the composition as a combined preparation for applying topically to the skin for preventing or treating bacterial, viral, parasitic or fungal infections or for reducing the number of bacterial, viral, parasitic or fungal organisms on the surface of the skin. In preferred embodiments, the composition is provided as two separate formulations in two individual containers intended for substantially concurrent administration. In preferred embodiments, one formulation contains a source of nitrite ions, and one formulation does not contain a source of nitrite ions.

Other aspects and advantages of the instant invention will be apparent from the following description, examples, and the appended claims.

### DETAILED DESCRIPTION OF THE INVENTION

Several powerful germicidal systems have been developed, in the last few decades, which are inherently unstable for extended time periods, and which must be deactivated prior to use by combining an oxy anion with a moderate-strength acid, to form the corresponding metastable acid form. Two such systems have emerged, based on either the chlorite or the nitrite system, where the basic activating reaction features:

Oxy anion+H+source→-ous acid

(chlorite[ClO₂] or nitrite[NO₂])+moderate strength acid→chlorous or nitrous Acid

Both chlorous acid and nitrous acid germicidal systems provide their antimicrobial activity as a direct result of the degradation (disproportionation) of these acids, whereby the transient, generally short-lived intermediate products destroy high levels of the target organisms through an oxidative mechanism. Both of these systems function effectively as topical germicides, but cannot function systemically since the functional pHs are significantly below pH 4. For topical application, it is advantageous to provide these systems in a thickened, adherent form. This may be accomplished by preparing relatively simple two-part gel systems, such as the various post-milking teat dips which have reached the market in recent years. Generally little problem is encountered in formulating the acid-containing gel phase, since there are a number of commercial gellants that are adequately stable at the requisite pH values. The task is more difficult, however, for the anion phase, and even simple thickeners for the oxidative anion phase are not readily available. As an example, Kross et al, U.S. Pat. No. 4,891,216 teaches a carbon-carbon backboned gellant, poly(acrylamido methanesulfonic acid (PAMS) as a stable material to formulate a thickened post-milking teat dip. Kross et al., U.S. Pat. No. 5,597,561 teaches a base polacrylamide polymer of the PAMS, which demonstrated similar properties. More recently, a post-milking teat dip comprising the acidified nitrite system has been disclosed U.S. patent application Ser. No. 10/575,326, wherein the nitrite phase relies on a similar carbon-backbone polymer for gelation, i.e., a PAMS-methacrylic acid copolymer. In all of these cases, the task of identifying a coherent, thickened topical system, particularly where the thickened anion phase, has to be thereafter compatible with the corresponding acid phase, was particularly facilitated by the full aqueous nature of all formulation components.

The presence of lipids, and other complex cosmetic ingredients into the intended formulation, significantly complicates the developmental process by further limiting the choice of thickening agents that can satisfy the multiple requirements of the desired formulation. Particular difficulty was encountered when attempting to formulate an effective. viscous antimicrobial hand lotion having a two-phase acid- and nitrite-ion composition, where the mixed composition was intended to provide the desirable attributes such as emolliency, humectancy, skin barrier formation, lubricity, and ease of mixture and application. The usual cosmetic agents familiar to those skilled in the art of such formulation were found to be unsuitable for developing an acceptable system.

In a first aspect, the present invention provides a composition, substantially a lotion for use primarily in the treatment of bacterial, viral, parasitic or fungal infections. The therapeutic and preventative qualities of the composition occur solely due to the active ingredient included in the formulation of the lotion. The composition embodiments of this invention are comprised of an oil-in-water emulsion which incorporates a simple and safe buffering system. Since the composition embodiments of this invention are an oil-in-water emulsion, the product can be easily removed from the skin or clothing by washing with soap and water. Additionally, the composition does not leave a greasy feeling on the skin. Still further, in some preferred embodiments of the invention, no fragrance is added to either the composition, such that the end-product has a substantially neutral olfactory sensation. The composition of this invention, substantially a lotion may be delivered to skin by means of, for instance, a wipe, a diaper, a spray from an aerosol or pump dispenser, a roll-on or a dabber. In use, the lotion embodiment of this invention is spread on sufficiently thick over the affected area to allow a good coating of the area to be protected. In use, the lotion embodiment of this invention is applied over the area to be protected which may be all or any portion of a skin surface, including the entire integumentary surface of a mammal or any portion thereof.

The concentration of the nitrite ion source may be up to 20% w/w, suitably 0.10 to 10%, preferably 0.25 to 1%. A particularly preferred concentration is 0.40% or 0.50% w/w.

The nitrite ions or source therefore are formulated in a pharmacologically acceptable carrier or diluent which may be an inert cream or ointment, such as the composition, substantially a lotion, of the instant invention. In a particular preferred form of the invention the acidifying agent and the source of nitrite ions or precursor therefore are separately disposed for admixture to release ions at the environment of use.

The composition, substantially a lotion, is preferably adapted for administration by a topical route. The composition for topical administration may be formulated as ointments, creams, suspensions, powders, solutions, pastes, gels, sprays, aerosols or oils. For treatment of the eye or other external tissues, for example mouth and skin, the compositions are preferably applied as a topical ointment or cream. When formulated in an ointment, the source of nitrite ions may be employed with either a paraffinic or a water-miscible ointment base. Alternatively, the source of nitrite ions may be formulated in a cream with an oil-in-water cream base or a water-in-oil base.

Such compositions may be formulated for human or for veterinary medicine. The present application should be interpreted as applying equally to humans as well as to animals, unless the context clearly implies otherwise.

In a second aspect, the invention provides a method for treating or preventing one or more of bacterial, viral, parasitic or fungal infections by applying an amount of a composition, substantially a lotion, according to the invention to the skin surface of a mammal, especially a human. Dosages of the composition, substantially a lotion, of the present invention can vary between wide limits, depending upon the disease or disorder to be treated, the severity of the condition, and the age and health of the individual to be treated, etc. and a physician will ultimately determine appropriate dosages to be used. In preferred embodiments, the number of organisms on a surface, such as, for instance, the skin are reduced on the order of at least 2 log₁₀, 3 log₁₀, 4 log₁₀, 5 log₁₀ or even 6 log₁₀ or 7 log₁₀. That is, the number of organisms on a surface, such as, for instance, the skin are reduced on the order of at least 50%, 60%, 70%, 80%, 90%, 95%, 98%, 99%, 99.5%, 99.9%, 99.99% or even 99.999%. In some instances, the number of organisms present on a surface, such as, for instance, the skin are reduced to a substantially undetectable level.

In some embodiments the subject organism is a bacteria, in some embodiments the subject organism is a virus, in some embodiments the subject organism is a yeast, and in some embodiments the subject organism is a fungus. The methods of the present invention may be useful against one, two, three, four, five or even ten or twenty or fifty or more identifiable microorganisms simultaneously. In some embodiments, the methods of the present invention are effective against at least half, at least two-thirds, at least three-quarters or even substantially all identifiable microorganisms simultaneously. Some representative organisms that the methods of the present invention may be useful in killing include, for instance, Escherichia coli, Staphylococcus aureus, Pseudomonas aeruginosa, and Candida albicans.

The methods of the present invention may be practiced by applying the compositions of the present invention directly to a surface. The surface may include the skin or mucosa, a medical device or implant or surgical instrument, or any surface desired to be sanitized or even sterilized. The compositions may be applied for any suitable length of time such as, for instance, at least 10 seconds, at least 30 seconds, at least 45 seconds, at least 1, 2, 3, 5, 7, 10, 15 or even 20 or 30 minutes, or the compositions may be applied to a surface and not removed at all until such time as they naturally wear off, evaporate, or are washed off. The compositions may be applied with a hand or the fingers, gloved or without gloves, or with a special applicator, or from a tube or bottle, etc. adapted to dispense the compositions.

In a third aspect, the invention provides a kit comprising a composition according to the invention housed in one or more containers together with instructions for using the composition as a combined preparation for applying topically to the skin for preventing or treating bacterial, viral, parasitic or fungal infections or for reducing the number of bacterial, viral, parasitic or fungal organisms on the surface of the skin. In preferred embodiments, the composition is provided as two separate formulations in two individual containers intended for substantially concurrent administration. In preferred embodiments, one formulation contains a source of nitrite ions, and one formulation does not contain a source of nitrite ions.

### EXAMPLE 1

A representative formula for the composition, substantially a lotion, of the invention is set forth in Table 1. In this embodiment, there are two separate formulations intended for use substantially concurrently for delivering and maintaining the stability of the active agent.

TABLE 1
LOTION CONCENTRATION INGREDIENT (Percent by weight)
Ingredients Listing (Cosmetic-Drug)
Hand Lotion-Blue

| | | Purpose(s) |
|---|---|---|
| Ingredient(s): | | |
| Allantoin | 0.50% | Skin protectant |
| Benzalkonium Chloride | 0.26% | Antiseptic |
| Water | QS to 100% | Solvent/diluent |
| Glycerin | 8.00% | Humectant/moisturizer |
| Benzyl Alcohol | 2.00% | Skin soother |
| Caprylic/Capric Triglyceride | 1.50% | Emollient |
| Acrylamide/Sodium | 3.25% | Polymer/colloid stabilizer |
| Acryloyldimethyltaurate | | |
| Copolymer | | |
| Lactic Acid | 1.25% | Acidifying agent |
| Isohexadecane | 0.50% | Slip/texture agent |
| Phenoxyethanol | 0.50% | Preservative |
| Polysorbate 80 | 0.50% | Surfactant |
| Methylparaben | 0.25% | Preservative |
| Citric Acid | 0.25% | Buffering agent |
| Ethylparaben | 0.10% | Preservative |
| Butylparaben | 0.05% | Preservative |
| Propylparaben | 0.05% | Preservative |
| Isobutylparaben | 0.05% | Preservative |
| Ascorbic Acid | 0.05% | Buffering agent |
| May Contain: | | |
| Blue (CI 42090) | 0.01% | Colorant |

TABLE 2
Ingredients Listing (Cosmetic-Drug)
Hand &Foot Lotion-Beige

| | | Purpose(s) |
|---|---|---|
| Ingredient(s) | | |
| Allantoin | 0.50% | Skin Protectant |
| Water | QS to 100% | Solvent/diluent |
| Butylene Glycol | 5.00% | Humectant/solvent |
| Dimethicone | 2.00% | Skin protectant |
| Polyacrylamide | 3.00% | Polymer/colloid stabilizer |
| Caprylic/Capric Triglyceride | 1.50% | Emollient |
| Magnesium Aluminum Silicate | 1.00% | Texture agent |
| C13-14 Isoparaffin | 1.00% | Slip/texture agent |
| Imidazolidinyl Urea | 0.50% | Preservative |
| Sodium Nitrite | 0.40% | Nitric Oxide source |
| Methylparaben | 0.25% | Preservative |
| Laureth-7 | 0.25% | Surfactant |
| Polyglyceryl-4 Isostearate | 0.20% | Surfactant |
| Cetyl PEG/PPG 10/1 | 0.20% | Surfactant |
| Dimethicone | | |
| Hexyl Laurate | 0.10% | Texture agent |
| Sodium Hydroxide | 0.05% | Buffering agent |
| May Contain: | | |
| Yellow 5 (CI 19140) | 0.01% | Colorant |
| Blue 1 (CI 42090) | 0.01% | Colorant |
| Red 40 (CI 16035) | 0.01% | Colorant |

### EXAMPLE 2

A representative formula for the composition, substantially a lotion, of the invention is set forth in Tables 3 and 4. In this embodiment, there are two separate formulations intended for use substantially concurrently for delivering and maintaining the stability of the active agent. Further is described an exemplary method and exemplary materials with which the formulation may be made.

TABLE 3

| Phase | INGREDIENT | % | weight |
|---|---|---|---|
| A | USP Purified Water | 81.095% | 137.862 |
| A | Winlet GL | 8.000% | 13.600 |
| A | Ascorbic Acid | 0.010% | 0.017 |
| A | Allantoin Powder | 0.500% | 0.850 |
| B | Unicol 50 | 0.520% | 0.884 |
| C | USP Purified Water | 0.262% | 0.445 |
| C | Citric Acid | 0.088% | 0.150 |
| D | Unichema LACA88S | 1.250% | 2.125 |
| D | Dermol M-5 | 1.500% | 2.550 |
| D | Unol BZAL | 2.000% | 3.400 |
| E | Simugel 600 | 3.750% | 6.375 |
| F | Phenonip | 1.000% | 1.700 |
| G | 1% FD&C Blue 1 | 0.025% | 0.043 |
| | (LCW#05601) in USP | | |
| | Purified Water | | |
| TOTAL | | 100.00% | 170.00 |

TABLE 4

| | | | Weight |
|---|---|---|---|
| | INGREDIENT | % | KG |
| A | USP Purified Water | 81.10% | 137.862 |
| A | Winlet GL | 8.00% | 13.600 |
| A | Ascorbic Acid | 0.01% | 0.017 |
| A | Allantoin Powder | 0.50% | 0.850 |
| B | Unicol 50 | 0.52% | 0.884 |
| C | USP Purified Water | 0.26% | 0.445 |
| C | Citric Acid | 0.09% | 0.150 |
| D | Unichema LACA88S | 1.25% | 2.125 |
| D | Dermol M-5 | 1.50% | 2.550 |
| D | Unol BZAL | 2.00% | 3.400 |
| E | Simugel 600 | 3.75% | 6.375 |
| F | Phenonip | 1.00% | 1.700 |
| G | 1% FD&C Blue 1 | 0.03% | 0.043 |
| | (LCW#05601) in USP Purified | | |
| | Water | | |
| | TOTAL | 100.00% | 170.00 |

Materials and methods. Record the time of each ingredient addition on a Manufacturing Log. All ingredients are to be added in the order they appear on the Manufacturing Log. Record the main batch tank number. Add USP Purified Water and Winlet GL, to the Main stainless steel, jacketed batch tank equipped with propeller and a sweep agitation. Increase the agitation and slowly sprinkle the Ascorbic Acid and Allantoin into vortex Set the temperature set point to 50 C and begin agitation. Add Unicol 50 to the batch tank and mix until uniform and clear. Lower set point to 40 C. In an appropriate vessel, Premix Phase C ingredients. Add all of formula amount of 25% Citric Acid solution, check and record pH. There is no pH adjustment, add 100% of the 25% Citric Acid solution and continue with the procedure. Record batch temperature. Increase agitation, add Unichema LACA88S, Dermol M-5, and Unol BZAL, mix until batch is uniform. Record batch temperature. Lower set point to 30 C. Increase agitation, add Simugel 600, mix until batch is uniform. Once batch is uniform mix an additional 60 minutes. Record batch temperature. Add Phenonip, mix until batch is uniform. Once batch is uniform mix an additional 10 minutes. Add the appropriate weights of the 1% FD&C Blue 1 (LCW#05601) in USP Purified Water Color Solution from the Initial Addition Column on the Batch.

Results. The ingredients of Table 4 were combined as shown in Table 4. The resultant lotion exhibited a pH of between 2.2 and 2.4. Tests conducted with a Brookfield viscometer at spindle 4, 4 rpm, disclosed a viscosity of 13,000-20,000 centipoise. The resultant lotion was acceptable for use on human skin. Under typical room temperature conditions the cream of this invention is a thick, viscous cream which is non-pourable. The Specific Gravity ranged between 0.96 and 1.02. CFTA or USP Microbiological testing revealed less than 100 cfu/g, no gram negatives and no pathogens. The lotion preferably appears blue in its dispenser.

### EXAMPLE 3

A representative formula for the composition, substantially a lotion, of the invention is set forth in Tables 5 and 6. In this embodiment, there are two separate formulations intended for use substantially concurrently for delivering and maintaining the stability of the active agent. Further is described an exemplary method and exemplary materials with which the formulation may be made.

TABLE 5

| INGREDIENT | % | weight | | Initial #1 |
|---|---|---|---|---|
| USP Purified Water | 62.580% | 62.580 | | |
| USP Purified Water | 0.075% | 0.075 | | |
| Sodium Hyrdroxide NF/FCC | 0.025% | 0.025 | | |
| Methylparaben | 0.300% | 0.300 | | |
| Allantoin Powder | 0.500% | 0.500 | | |
| Abil WE 09 | 0.200% | 0.200 | | |
| Dimethicone 200/100 | 2.000% | 2.000 | | |
| Dermol M-5 | 1.500% | 1.500 | | |
| Sepigel 305 | 4.000% | 4.000 | | |
| USP Purified Water | 0.600% | 0.600 | | |
| Sodium nitrite GR FCC | 0.400% | 0.400 | | |
| FREE FL 030002-S140 | | | | |
| USP Purified Water | 20.000% | 20.000 | | |
| Winlet BG | 5.000% | 5.000 | | |
| Veegum Granules | 1.000% | 1.000 | | |
| USP Purified Water | 1.000% | 1.000 | | |
| Germall 115 | 0.500% | 0.500 | | |
| 1% FD&C Red 40 in USP | 0.120% | 0.120 | | |
| Purified Water | | | | |
| 1% FD&C Blue 1 in USP | 0.040% | 0.040 | | |
| Purified Water | | | | |
| 1% FD&C Yellow 5 in USP | 0.160% | 0.160 | | |
| Purified Water | | | | |
| TOTAL | 100.00% | 100.00 | kg | |

TABLE 6

| | | | | Weight |
|---|---|---|---|---|
| Phase | DMI # | INGREDIENT | % | KG |
| A | 19000 | USP Purified Water | 62.580% | 62.580 |
| B | 19000 | USP Purified Water | 0.075% | 0.075 |
| B | 17670 | Sodium Hyrdroxide NF/FCC | 0.025% | 0.025 |
| C | 15070 | Methylparaben | 0.300% | 0.300 |
| C | 10140 | Allantoin Powder | 0.500% | 0.500 |
| D | 16795 | Abil WE 09 | 0.200% | 0.200 |
| E | 11691 | Dimethicone 200/100 | 2.000% | 2.000 |
| E | 11028 | Dermol M-5 | 1.500% | 1.500 |
| F | 16747 | Sepigel 305 | 4.000% | 4.000 |
| G | 19000 | USP Purified Water | 0.600% | 0.600 |
| G | 17683* | Sodium nitrite GR FCC FREE | 0.400% | 0.400 |
| | | FL 030002-S140 | | |
| H | 19000 | USP Purified Water | 20.000% | 20.000 |
| J | 10700 | Winlet BG | 5.000% | 5.000 |
| J | 15053 | Veegum Granules | 1.000% | 1.000 |
| K | 19000 | USP Purified Water | 1.000% | 1.000 |
| K | 13650 | Germall 115 | 0.500% | 0.500 |
| L | D145 | 1% FD&C Red 40 in USP | 0.120% | 0.120 |
| | | Purified Water | | |
| L | D134 | 1% FD&C Blue 1 in USP | 0.040% | 0.040 |
| | | Purified Water | | |
| L | D144 | 1% FD&C Yellow 5 in USP | 0.160% | 0.160 |
| | | Purified Water | | |
| | | TOTAL | 100.000% | 100.000 |

Materials and methods. Record the main batch tank number. Add Phase A water to the Main stainless steel, jacketed batch tank equipped with propeller and a sweep agitation. Set the temperature set point to 60 C and begin agitation. In an appropriate vessel, Premix USP Purified water and Sodium Hyrdroxide NF/FCC. Mix until a uniform solution is obtained. Once USP Purified water and Sodium Hyrdroxide NF/FCC is uniform and clear add to the batch. Increase agitation and slowly sprinkle Methylparaben and Allantoin powder to the batch. Add Abil WE 09 into the vortex of the main batch tank and mix until uniform. After the Abil WE 09 addition the steam should be shut off and not turned on again for the remainder of the batch. Record batch temperature. Increase agitation, and add Dimethicone 200/100 and Dermol M-5, mix until batch is uniform. Once batch is uniform mix an additional 10 minutes. Record batch temperature. Increase agitation, add Sepigel 305, mix until batch is uniform. Once batch is uniform mix for a minimum of an additional 30 minutes. This is corrosive material, when handling Sodium nitrite powder and solution, use caution. Wear gloves and mask. If a spill occurs on skin and clothing, remove clothes immediately and rinse with water for 10 minutes. In an appropriate vessel, Premix USP Purified Water and Sodium nitrite GR FCC FREE FL 03002-S140. Mix until a clear solution is obtained. Once USP Purified Water and Sodium nitrite GR FCC FREE FL 03002-S140 is clear lower batch agitation and add USP Purified Water and Sodium nitrite GR FCC FREE FL 03002-S140 to batch slowly, in increments, and monitor viscosity and agitation closely, a large drastic drop in viscosity will occur. Add the Phase H water to an appropriate propeller agitated jacketed auxiliary vessel. Heat the water to 60 C. Premix Winlet BG and the Veegum. Mix the Winlet and Veegum until a uniform slurry is obtained. Once the slurry is uniform preheat the USP Purified Water to 65 C. Turn off the steam and do not turn the steam back on. Add the Winlet BF and Veegum Granules premix to the tank, once this is uniform, mix for an additional 30 minutes. Once the 30 minute mix period is complete and the phase is below 50 C it may be added to the main batch tank. Once the premix is below 50 C add the premix to the main batch tank and mix for 30 minutes. At the end of the 30 minute mix period continue to the next step. Premix the USP Purified water and Germall 115, once a solution is obtained and when the batch has cooled to 40 C or below, slowly add Phase H USP Purified Water to the batch. Purge the bottom valve. Mix until uniform. Then mix an additional 10 minutes. Add the appropriate weights of the 1% FD&C Red 40 in USP Purified Water, 1% FD&C Blue 1 in USP Purified Water, and 1% FD&C Yellow 5 in USP Purified Water Color Mixes from the Initial Addition Column on the Batch Adjustment Worksheet. Allow the batch to mix for 15 minutes. After the mix period, purge the bottom valve. If more color additions are needed, add the appropriate quantities indicated on the Shade Adjustment Worksheet. Allow batch to mix for 10 minutes between additions. Continue this procedure until the shade is approved.

Results. The ingredients of Table 6 were combined as shown in Table 6. The resultant lotion exhibited a pH of between 7.3 and 7.6. Tests conducted with a Brookfield viscometer at spindle 4, 4 rpm, disclosed a viscosity of 100,000-150,000 centipoise. The resultant lotion was acceptable for use on human skin. Under typical room temperature conditions the cream of this invention is a thick, viscous cream which is non-pourable. The Specific Gravity ranged between 0.96 and 1.02. CFTA or USP Microbiological testing revealed less than 100 cfu/g, no gram negatives and no pathogens. The lotion preferably appears beige in its dispenser.

### EXAMPLE 4

### Microbial Challenge of Hand Lotion Preparations

Materials and Methods.

Bacteria were plated on Trypticase Soy Agar and incubated at 35-37° C., for 18-24 hours. They were subsequently plated a second time and again incubated at 35-37° C., for 18-24 hours. Yeasts were plated on Sabouraud Dextrose Agar and incubated at 25-30° C., for 24-48 hours. They were subsequently plated a second time and again incubated at 25-30° C., for 24-48 hours. A 0.5 McFarland suspension. (approximately 5.0×10⁸ CFU/ml for bacteria, 5.0×10⁷ CFU/ml for yeast) was prepared in sterile saline.

Test mixtures were prepared by mixing 1 ml of the microorganism suspension with 9.0 grams of the test sample. In cases in which the test sample was a combination of two components, 4.5 grams of the first component were mixed with 4.5 grams of the second component. These were then mixed with a sterile wooden tongue depressor and then vortexed. This procedure was found to produce a uniform mixture. Following the addition of the microorganism suspension, the mixture was vortexed for 15 seconds.

At the appropriate time interval, 2.0 grams of the mixtures were added to 18 ml of D/E Neutralization Broth. A further 1/10 dilution of the D/E broth in saline was prepared. Five 2.0 ml samples of the D/E broth were added to petri plates. Duplicate 1.0 ml samples were added to petri plates, and duplicate 1.0 ml samples of the 1/10 dilution were added to petri plates. Approximately 10 ml of liquid Trypticase Soy Agar were added to the each bacterial petri plate and approximately 10 ml of liquid Sabouraud Dextrose Agar were added to the each yeast petri plate. Plates were incubated at room temperature and allowed to solidify.

Bacterial plates were incubated at 35-37° C., for 24-48 hours, and yeast plates were incubated at 25-30° C., for 48-72 hours. Colony forming units were counted. A control study was run for each microorganism, in which a sample of saline was challenged, instead of the test sample.

In order to assess the neutralization ability of the D/E broth, 2 μl of the microorganism suspensions were added to 2 ml of several of the 1 minute D/E broth samples. 1 μl aliquots of this mixture were placed in a petri dish and the appropriate liquid agar was added. Plates were incubated at room temperature and allowed to solidify.

Bacterial plates were incubated at 35-37° C., for 24-48 hours, and yeast plates were incubated at 25-30° C., for 48-72 hours. Colony forming units were counted. Equal quantities of a composition according to Table 5 or 6 and Table 3 or 4 were mixed and the pH was measured. Equal quantities of a composition according to Table 5 or 6 with no benzyl alcohol and no ascorbic acid; and a composition according to Table 3 or 4 were mixed and the pH was measured. Equal quantities of a composition according to Table 3 or 4 with no ascorbic acid; and a composition according to Table 3 or 4 were mixed and the pH was measured. The pH of an actual sample of a composition according to Table 3 or 4 and a composition according to Table 5 or 6 that was mixed with C. albicans was measured.

The samples were tested against E. coli ATCC 8739. The results indicated a greater than 7.11 log reduction in the numbers of organisms after 1 and after 2 minutes when contacted with a composition according to Table 3 or 4 in combination with a composition according to Table 5 or 6. This was true whether or not ascorbic acid was present in the composition according to Table 3 or 4.

The samples were tested against S. aureus ATCC 29213. The results indicated a greater than 7.80 log reduction in the numbers of organisms after 1 and after 2 minutes when contacted with a composition according to Table 3 or 4 in combination with a composition according to Table 5 or 6. This was true whether or not ascorbic acid was present in the composition according to Table 3 or 4.

The samples were tested against P. Aeruginosa ATCC 10145. The results indicated a greater than 7.15 log reduction in the numbers of organisms after 1 and after 2 minutes when contacted with a composition according to Table 3 or 4 in combination with a composition according to Table 5 or 6. This was true whether or not ascorbic acid was present in the composition according to Table 3 or 4.

The samples were tested against C. Albicans ATCC 10231. The results indicated a greater than 5.30 log reduction in the numbers of organisms after 1 and after 2 minutes when contacted with a composition according to according to Table 5 or 6.

The pH of each sample was analyzed and found to be 3.4 for a composition according to Table 5 or 6; 3.42 for a composition according to Table 3 or 4 in combination with a composition according to Table 5 or 6 lacking Ascorbic Acid and Benzyl Alcohol; 3.49 for a composition according to Table 5 or 6 lacking Ascorbic Acid in combination with a composition according to Table 3 or 4; and 3.50 for a composition composition according to Table 3 or 4 in combination with a composition according to Table 5 or 6.

### EXAMPLE 5

### Microbial Challenge of Hand Lotion Preparations

Materials and Methods.

Yeasts were plated on Sabouraud Dextrose Agar and incubated at 25-30° C., for 24-48 hours. They were subsequently plated a second time and again incubated at 25-30° C., for 24-48 hours. A 0.5 McFarland suspension (approximately 5.0×10⁶ CFU/ml) was prepared in sterile saline.

Test mixtures were prepared by mixing 1 ml of the microorganism suspension with 9.0 grams of the test sample. In the first set of experiments, 4.5 grams of Sample A, B or C was added to 4.5 grams of Sample D. In the second set of experiments, 4.5 grams of Sample E was added to 4.5 grams of Sample F. pH measurements were made of these samples. These samples were then mixed with a sterile wooden tongue depressor and then vortexed. This procedure was found to produce a uniform mixture. Following the addition of the microorganism suspension, the mixture was vortexed for 15 seconds.

At one minute, two minutes, five minutes and ten minutes; 2.0 grams of the mixture was added to 18 ml of D/E Neutralization Broth. A further 1/10 dilution of the D/E broth in saline was prepared. Five 2.0 ml samples of the D/E broth were added to petri plates. Duplicate 1.0 ml samples were added to petri plates, and duplicate 1.0 ml samples of the 1/10 dilution were added to Petri plates. Approximately 10 ml of liquid Sabouraud Dextrose Agar were added to each petri plate. Plates were incubated at room temperature and allowed to solidify. They were then incubated at 25-30° C., for 48-72 hours. Colony forming units were counted.

A control study was run in which a sample of saline was challenged, instead of the test sample.

In order to assess the neutralization ability of the D/E broth, 2 μl of the microorganism suspension was added to 2 ml of the 10 minute 37-21-01 (Beige)+37-25-01 (Acid without BAC) pH adjusted DIE broth sample. A 10 ul aliquot of this mixture was placed in a petri dish and Sabouraud Dextrose Agar was added. Plates were incubated at room temperature and allowed to solidify.

The sample compositions were tested for the ability to kill C. albicans ATCC 10231. A composition according to Table 5 or 6 (but having no nitrite) in combination with a composition according to Table 3 or 4 demonstrated limited killing activity even ten minutes after application.

A composition according to Table 5 or 6 (but containing no Germall or Allantoin) in combination with a composition according to Table 3 or 4 demonstrated a time dependent rate of killing the yeast. A composition according to Table 5 or 6 (but containing no Germall) in combination with a composition according to Table 3 or 4 demonstrated a time dependent rate of killing the yeast. A composition according to Table 5 or 6 in combination with a composition according to Table 3 or 4 (but without Benzalkonium Chloride) demonstrated a time dependent rate of killing the yeast, although the rate was slower than with other compounds. When the pH of a mixture of a composition according to Table 5 or 6 in combination with a composition according to Table 3 or 4 (but without Benzalkonium Chloride) was adjusted to 3.20, the rate of kill was greatly improved. Specifically, a composition according to Table 5 or 6 in combination with a composition according to Table 3 or 4 whether or not Germall or Allantoin was present provided at least a log 5.08 reduction in organisms after 5 minutes of exposure. A composition according to Table 5 or 6 in combination with a composition according to Table 3 or 4 (but without Benzalkonium Chloride) provided a 2.94 and 3.62 reduction in the number of organisms after 5 and 10 minutes, respectively. A composition according to Table 5 or 6 in combination with a composition according to Table 3 or 4 (but without Benzalkonium Chloride) provided a 4.3.0 and at least 5.08 reduction in the number of organisms after 5 and 10 minutes, respectively, if the pH was adjusted to 3.20.

### EXAMPLE 6

### Summary

This study used a modification of the test methods outlined in a Tentative Final Monograph (Federal Register, 22 Jul. 1991, vol. 56: 140; p. 33678-33680) to evaluate the bactericidal properties of one (1) first aid antiseptic product when challenged with three (3) species: Escherichia coli (ATCC #8739), Pseudomonas aeruginosa (ATCC #9027), and Staphylococcus aureus (ATCC #6538). The percent and Log₁₀ reductions from the initial population of each challenge species were determined following a ten (10) minute exposure to the test product at 32±2° C. in the presence of 10% (v/v) serum. Under these test conditions, a minimum reduction of three (3) Log₁₀ has been specified by the Monograph as the criterion for approval of a drug product as an effective first aid antiseptic. The test product met the acceptance criterion specified by the Monograph, reducing microbial populations of all three (3) challenge strains by more than 7.0 Log₁₀ following a ten (10) minute exposure at 32±2° C. in the presence of 10% (v/v) serum.

Materials and methods.

This study used an In-Vitro Time-Kill Method to evaluate the bactericidal properties of one (1) test product, On The Job Men's Antimicrobial Hand Lotion (Lot Number B6613/B6620), when challenged with Escherichia coli (ATCC #8739), Pseudomonas aeruginosa (ATCC #9027), and Staphylococcus aureus aureus (ATCC #6538). The percent and Log₁₀ reductions from the initial population of each challenge species were determined following ten (10) minute exposures to the test product at 32°±2° C. in the presence of 0% (v/v) serum. All agar-plating was performed in duplicate. The Study Protocol, included as Addendum I of this Final Report, presents the study methodology in detail, as do General Data Gathering Forms (Form No. 91-L-002) in Addenda V and VI of this Final Report. One (1) deviation from BioScience Laboratories, Inc., Standard Operating Procedure L-2059 occurred (reference Section 11.0 of this Final Report), and as is detailed on a Protocol and/or SOP Deviation Recording Form (Form No. 99-QA-004) in Addendum I of this Final Report, it had no adverse effect upon the study outcome.

Results

The population of each broth culture used for the Bactericidal Assay procedure was demonstrated to be greater than 1×10⁹ CFU/mL. The neutralizing solution (DeylEngley Broth (D/E) and plating medium (Tryptic Soy Agar with product neutralizers (TSA+) were demonstrated to be effective in neutralizing the antibacterial properties of the test product when challenged with each microorganism strain. Upon challenge with each microorganism, the neutralizing solution (D/E) and plating medium (TSA+) were also demonstrated to be non-toxic to each species. The results of the Neutralizer Efficacy and Neutralizer Toxicity Evaluations are presented in Table 8 and Table 9, respectively.

Table 7 presents the Log₁₀ Reductions and Percent Reductions produced by the Test Product versus each of the three (3) challenge microorganisms.

TABLE 7
Bactericidal Assay Results

| Challenge | | Exposure | Replicate | Log₁₀ | Percent |
|---|---|---|---|---|---|
| Microorganism | Population | Time | # | Reduction | Reduction |
| Escherichia coli | 2.2850 × 10¹⁰ | 10 minutes | 1 | 8.3589 | 99.9999% |
| (ATCC #8739) | | | 2 | 8.3589 | 99.9999% |
| | | | 3 | 8.3589 | 99.9999% |
| Pseudomonas aerugin. | 5.050 × 10⁹ | 10 minutes | 1 | 7.7033 | 99.9999% |
| (ATCC #9027) | | | 2 | 7.7033 | 99.9999% |
| | | | 3 | 7.7033 | 99.9999% |
| Staphylococcus | 6.660 × 10¹⁰ | 10 minutes | 1 | 8.8235 | 99.9999% |
| (ATCC #6538) | | | 2 | 8.8235 | 99.9999% |
| | | | 3 | 8.8235 | 99.9999% |

TABLE 8
Neutralizer Efficacy Results

| | Rep- | Control Population | | Test Population | |
|---|---|---|---|---|---|
| Challenge | li- | (WFI) | | (Test Product) | |
| Microorganism | cate # | CFU/mL | Log_{1O} | CFU/mL | Log₁₀ |
| Escherichia coli | 1 | 6.050 × 10⁷ | 7.7818 | 4.050 × | 7.6075 |
| (ATCC #8739) | 2 | 5.950 × 10⁷ | 7.7745 | 5.00 × 10⁷ | 7.6990 |
| | 3 | 5.850 × 10⁷ | 7.7672 | 5.050 × 10⁷ | 7.7033 |
| Pseudomonas | 1 | 3.850 × 10⁷ | 7.5855 | 6.90 × 10⁷ | 7.8388 |
| (ATCC #9027) | 2 | 5.950 × 10⁷ | 7.7745 | 4.950 × 10⁷ | 7.6946 |
| | 3 | 6.150 × 10⁷ | 7.7889 | 5.050 × 10⁷ | 7.7033 |
| Staphylococcus | 1 | 9.950 × 10⁷ | 7.9978 | 8.450 × 10⁷ | 7.9269 |
| (ATCC #6538) | 2 | 9.650 × 10⁷ | 7.9845 | 9.70 × 10⁷ | 7.9868 |
| | 3 | 1.010 × 10⁸ | 8.0043 | 9.150 × 10⁷ | 7.9614 |

TABLE 9
Neutralizer Toxicity Results

| | | Control population | | Test Population | |
|---|---|---|---|---|---|
| | Rep- | (without | | (with neutralizer; | |
| Challenge | li- | neutralizer) | | D/E Broth) | |
| Microorganism | cate # | CFU/mL | Log₁ | CFU/m | Log_{1O} |
| Escherichia coli | 1 | 6.40 × 10⁸ | 8.8062 | 6.150 × 10⁸ | 8.7889 |
| (ATCC #8739) | 2 | 8.30 × 10⁸ | 8.9191 | 6.650 × 10⁸ | 8.8228 |
| | 3 | 7.10 × 10⁸ | 8.8513 | 6.150 × 10⁸ | 8.7889 |
| Pseudomonas | 1 | 5.950 × 10⁸ | 8.7745 | 6.10 × 10⁸ | 8.7853 |
| (ATCC #9027) | 2 | 5.350 × 10⁸ | 8.7284 | 5.80 × 10⁸ | 8.7634 |
| | 3 | 7.150 × 10⁸ | 8.8543 | 6.250 × 10⁸ | 8.7959 |
| Staphylococcus | 1 | 1.0050 × | 9.0022 | 8.350 × 10⁸ | 8.9217 |
| aureus | 2 | 8.60 × 10⁸ | 8.9345 | 9.60 × 10⁸ | 8.9823 |
| (ATCC #6538) | 3 | 1.0050 × 10⁹ | 9.0022 | 1.0150 × 10⁹ | 9.0065 |

### EXAMPLE 7

### Summary

The objective of this study was to determine the irritation and/or sensitization potential of the test article after repeated application under occlusive patch test conditions to the skin of human subjects (non-exclusive panel).

Materials and methods

A total of 54 subjects, 11 males and 43 females ranging in age from 18 to 70 years, were empaneled for this test.

The subjects chosen were dependable and able to read and understand instructions. The subjects did not exhibit any physical or dermatological condition that would have precluded application of the test article or determination of potential effects of the test article. The 9 Repeated Insult (occlusive) Patch Test (9-RIPT) was conducted as follows:

A sufficient amount of the test article (an amount to adequately cover the surface of the patch unit—approximately 0.1 g-0.15 g) was placed onto a Parke-Davis Readi-Bandage® occlusive patch, which was applied to the back of each subject between the scapulae and waist, adjacent to the spinal mid-line. This procedure was performed by a trained technician/examiner and repeated every Monday, Wednesday and Friday until 9 applications of the test article had been made.

The subjects were instructed to remove the patch 24 hours after application. Twenty-four hour rest periods followed the Tuesday and Thursday removals and 48-hour rest periods followed each Saturday removal. Subjects returned to the Testing Facility and the site was scored by a trained examiner just prior to the next patch application.

If a subject developed a positive reaction of a level 2 erythema or greater during the Induction phase or if, at the discretion of the Study Director, the skin response warranted a change in site, the patch was applied to a previously unpatched, adjacent site for the next application. If a level 2 reaction or greater occurred at the new site, no further applications were made. However, any reactive subjects were subsequently Challenge patch tested.

After a rest period of approximately 2 weeks (no applications of the test article), the Challenge patch was applied to a previously unpatched (virgin) test site. The site was scored 24 and 72 hours after application. All subjects were instructed to report any delayed skin reactivity that occurred after the final Challenge patch reading. When warranted, selected test subjects were called back to the Clinic for additional examinations and scoring to determine possible increases or decreases in Challenge patch reactivity.

Dermal responses for both the Induction and Challenge phases of the study were scored according to the following 6-point scale: 0=No evidence of any effect; +=Barely perceptible (Minimal, faint, uniform or spotty erythema); 1=Mild (Pink, uniform erythema covering most of the contact site); 2=Moderate (Pink-red erythema uniform in the entire contact site); 3=Marked (Bright red erythema with/without petechiae or papules); and 4=Severe (Deep red erythema with/without vesiculation or weeping).

All other observed dermal sequelae (eg, edema, dryness, hypo- or hyperpigmentation) were appropriately recorded on the data sheet and described as mild, moderate or severe.

Results

Fifty-two (52/54) subjects satisfactorily completed the test procedure on the test Hand Lotion. One (1/54) subject discontinued for personal reasons unrelated to the conduct of the study. One (1/54) subject discontinued due to a moderate tape reaction. Discontinued panelist data are shown up to the point of discontinuation, but are not used in the Conclusions section of this final report. There was no skin reactivity observed at any time during the course of the study. Under the conditions of a repeated insult (occlusive) patch test procedure conducted in 52 subjects, the test Hand Lotion was found to not induce skin irritation nor show any evidence of induced allergic contact dermatitis in

While the composition, substantially a lotion, herein described constitutes preferred embodiments of this invention, it is to be understood that the invention is not limited to either precise formulation and that changes may be made therein without departing from the scope of the invention which is defined in the appended claims.

We claim:

1. A composition comprising a skin protectant present in a concentration of about 0.50 to 2.50% by weight, a Humectant/moisturizer present in a concentration of about 5 to 8% by weight, an Emollient present in a concentration of about 1 to 2% by weight, a polymer/colloid stabilizer present in a concentration of about 3.0 to 4.0% by weight, a Slip/texture agent present in a concentration of about 0.50 to 2.50% by weight, a Preservative present in a concentration of about 0.75 to 1.00% by weight, a Surfactant present in a concentration of about 0.40 to 0.75% by weight, a Buffering agent present in a concentration of about 0.40 to 0.75% by weight, optionally an Antiseptic present in a concentration of about 0.25 to 0.75% by weight, optionally a skin soother present in a concentration of about 0.50 to 2.00% by weight, optionally an acidifying agent present in a concentration of about 1.00 to 2.00% by weight, optionally a source of nitrite ions present in a concentration of about 0.40 to 1.00% by weight, optionally a colorant present in a concentration of about 0.01 to 0.05% by weight.

2. A composition according to claim 1 wherein at least two distinct and separate formulations are provided housed separately, and wherein a first formulation contains the source of nitrite ions and a second formulation does not contain substantial amounts of the source of nitrite ions.

3. A composition according to claim 2 wherein the first formulation comprising a source of nitrite ions comprises a Skin protectant present in a concentration of about 0.50 to 2.50% by weight, a Humectant/moisturizer present in a concentration of about 5 to 8% by weight, an Emollient present in a concentration of about 1 to 2% by weight, a polymer/colloid stabilizer present in a concentration of about 3.0 to 4.0% by weight, a Slip/texture agent present in a concentration of about 0.50 to 2.50% by weight, a Preservative present in a concentration of about 0.75 to 1.00% by weight, a Surfactant present in a concentration of about 0.40 to 0.75% by weight, a Buffering agent present in a concentration of about 0.40 to 0.75% by weight, a source of nitrite ions present in a concentration of about 0.25 to 2.00% by weight, and a colorant present in a concentration of about 0.01 to 0.05% by weight.

4. A composition according to claim 2 wherein the second formulation having no substantial amount of the source of nitrite ions comprises a Skin protectant present in a concentration of about 0.50 to 2.50% by weight, a Humectant/moisturizer present in a concentration of about 5 to 8% by weight, an Emollient present in a concentration of about 1 to 2% by weight, a polymer/colloid stabilizer present in a concentration of about 3.0 to 4.0% by weight, a Slip/texture agent present in a concentration of about 0.50 to 2.50% by weight, a Preservative present in a concentration of about 0.75 to 1.00% by weight, a Surfactant present in a concentration of about 0.40 to 0.75% by weight, a Buffering agent present in a concentration of about 0.40 to 0.75% by weight, an Antiseptic present in a concentration of about 0.25 to 0.75% by weight, a skin soother present in a concentration of about 0.50 to 2.00% by weight, an acidifying agent present in a concentration of about 1.00 to 2.00% by weight, and a colorant present in a concentration of about 0.01 to 0.05% by weight.

5. A method for treating or preventing one or more of bacterial, viral, parasitic or fungal infections comprising applying a pharmaceutically effective amount of a composition according to claim to the skin surface of a mammal.

6. A method for reducing the number of bacterial, viral, parasitic or fungal organisms on the surface of the skin comprising applying a pharmaceutically effective amount of a composition according to claim to the skin surface of a mammal.

7. A kit comprising a composition according to claim 2 housed separately in one or more containers together with instructions for using the composition as a combined preparation for applying topically to the skin for preventing or treating bacterial, viral, parasitic or fungal infections or for reducing the number of bacterial, viral, parasitic or fungal organisms on the surface of the skin.

8. A kit according to claim 7 wherein the composition is provided as two separate formulations in two individual containers intended for substantially concurrent administration.

9. A kit according to claim 7 wherein a first formulation contains a source of nitrite ions, and a second formulation does not contain a source of nitrite ions.
